(19) United States
(12) Patent Application Publication
(10) Pub. No.: US 2022/0362049 A1
(43) Pub. Date: Nov. 17, 2022

(54) FLUID COLLECTION ASSEMBLIES INCLUDING ONE OR MORE FLUID FLOW FEATURES

(71) Applicant: PureWick Corporation, EI Cajon, CA (US)

(72) Inventors: Nick Austermann, Atlanta, GA (US); Ashley Marie Johannes, Atlanta, GA (US)

(21) Appl. No.: 17/628,411

(22) PCT Filed: Jul. 22, 2020
(86) PCT No.: PCT/US2020/043059

§ 371 Date: Jan. 19, 2022

Related U.S. Application Data
(60) Provisional application No. 62/877,558, filed on Jul. 23, 2019.

(51) Int. Cl. A61F 5/44; A61B 10/00; A61F 5/451; A61F 5/453; A61F 5/455

(52) U.S. Cl. CPC A61F 5/4401; A61B 10/007; A61F 5/4404; A61F 5/451; A61F 5/453; A61F 5/455

(57) ABSTRACT

An example fluid collection assembly includes a fluid impermeable barrier defining at least one exterior surface. The fluid impermeable barrier includes at least one interior surface defining a chamber and an exterior surface opposite the interior surface. The fluid impermeable barrier defines at least one opening that is configured to be positioned adjacent to a urethral opening that allows fluids (e.g., urine) to enter the chamber. The fluid impermeable barrier also includes one or more fluid flow features formed in or on the exterior surface of the fluid impermeable barrier. The fluid flow features are configure to facilitate removal of one or more fluids between the exterior surface and the skin.

### CROSS-REFERENCE TO RELATED APPLICATIONS

This application claims priority to U.S. Provisional Application No. 62/877,558 filed on Jul. 23, 2019, the entire disclosure of which is incorporated herein by this reference.

### BACKGROUND

A person or animal may have limited or impaired mobility such that typical urination processes are challenging or impossible. For example, a person may experience or have a disability that impairs mobility. A person may have restricted travel conditions such as those experienced by pilots, drivers, and workers in hazardous areas. Additionally, sometimes urine collection is needed for monitoring purposes or clinical testing.

Urinary catheters, such as a Foley catheter, can be used to address some of these circumstances, such as incontinence. Unfortunately, urinary catheters can be uncomfortable, painful, and can lead to complications, such as infections. Additionally, bed pans, which are receptacles used for the toileting of bedridden patients are sometimes used. However, bedpans can be prone to discomfort, spills, and other hygiene issues.

### SUMMARY

In an embodiment, a fluid collection assembly is disclosed. The fluid collection assembly includes a fluid impermeable barrier defining a chamber, at least one opening configured to be positioned at least proximate to a urethral opening of an individual, and an outlet. The fluid impermeable barrier includes at least one exterior surface and at least one interior surface defining the chamber. The fluid collection assembly also includes one or more fluid flow features at least one of defined by, formed by, or disposed on at least a portion of the fluid impermeable barrier or on at least a portion of the at least one exterior surface of the fluid impermeable barrier. The one or more fluid flow features are configured to at least one of remove trapped fluids between the at least one exterior surface and skin of the individual.

In an embodiment, a method of using a fluid collection assembly is disclosed. The method includes disposing at least one opening defined by a fluid impermeable barrier of the fluid collection assembly at least proximate to a urethral opening of an individual. The fluid impermeable barrier defines an outlet. The fluid impermeable barrier includes at least one exterior surface and at least one interior surface defining a chamber. The at least one opening and the outlet provide access to the chamber. The method also includes, with one or more fluid flow features, removing trapped fluids from between the at least one exterior surface of the fluid impermeable barrier and skin of the individual to a location that is not between the at least one exterior surface of the fluid impermeable barrier and the skin of the individual. The one or more fluid flow features are at least one of defined by, formed by, or disposed on at least a portion of the fluid impermeable barrier or on at least a portion of the at least one exterior surface.

In an embodiment, a fluid collection system is disclosed. The fluid collection system includes a fluid collection assembly. The fluid collection assembly includes a fluid impermeable barrier defining a chamber, at least one opening configured to be positioned at least proximate to a urethral opening of an individual, and an outlet. The fluid impermeable barrier includes at least one exterior surface and at least one interior surface defining the chamber. The fluid collection assembly also includes one or more fluid flow features at least one of defined by, formed by, or disposed on at least a portion of the fluid impermeable barrier or on at least a portion of the at least one exterior surface of the fluid impermeable barrier. The one or more fluid flow features are configured to at least one of remove trapped fluids between the at least one exterior surface and skin of the individual. The fluid collection system also includes a fluid storage container and a vacuum source configured to apply a suction force. The fluid collection system further includes at least one tube that places the chamber, the fluid storage container, and the vacuum source in fluid communication with each other.

Features from any of the disclosed embodiments may be used in combination with one another, without limitation. In addition, other features and advantages of the present disclosure will become apparent to those of ordinary skill in the art through consideration of the following detailed description and the accompanying drawings.

### BRIEF DESCRIPTION OF THE DRAWINGS

The drawings illustrate several embodiments of the present disclosure, wherein identical reference numerals refer to identical or similar elements or features in different views or embodiments shown in the drawings.

FIG. 1A is a schematic isometric view of a fluid collection assembly, according to an embodiment.

FIG. 1B is a cross-sectional view of the fluid collection assembly shown in FIG. 1A, according to an embodiment.

FIGS. 2-4 are isometric views of fluid collection assemblies that include one or more recesses following different paths, according to different embodiments.

FIGS. 5-7 are schematic cross-sectional views of a portion of a fluid collection assembly that includes fluid flow features other than recesses, according to different embodiments.

FIG. 8 is a schematic cross-sectional view of a fluid collection assembly that is configured to received urine from a male urethral opening, according to an embodiment.

FIG. 9 is a schematic illustration of fluid collection system that includes a fluid collection assembly, according to an embodiment.

### DETAILED DESCRIPTION

Embodiments disclosed herein include a fluid collection assembly, systems including the same, and method of using the same. An example fluid collection assembly includes a fluid impermeable barrier defining at least one exterior surface. The fluid impermeable barrier includes at least one interior surface defining a chamber and an exterior surface opposite the interior surface. The fluid impermeable barrier defines at least one opening (e.g., elongated opening) that is configured to be positioned adjacent to a urethral opening (e.g., female urethral opening) that allows fluids (e.g., urine) to enter the chamber. The fluid impermeable barrier also includes one or more fluid flow features formed in or on the exterior surface of the fluid impermeable barrier. The fluid flow features are configure to facilitate removal of one or more fluids between the exterior surface and the skin.

During use, the fluid collection assembly is positioned such that the opening defined by the fluid impermeable barrier is positioned adjacent to the urethral opening of the individual. Positioning the opening to be adjacent the urethral opening causes portions of the fluid impermeable barrier to contact or be positioned proximate (e.g., at most about 1 mm, at most about 2 mm, at most about 3 mm, at most about 5 mm, or at most about 10 mm) to the skin of the individual. For brevity and clarity, the portions of the fluid impermeable barrier that contact or are positioned proximate to the skin of the individual are referred to as “contact portions.” The remainder of the fluid impermeable barrier (e.g., the non-contact portions of the fluid impermeable barrier) are considered spaced from the skin of the individual and, for brevity and clarity, are referred to as “spaced portions.” The exact portions of the fluid impermeable barrier that form the contact and spaced portions of the fluid impermeable barrier may depend on a number of factors, including the position of fluid collection assembly on the individual (i.e., the position of the fluid collection assembly relative to the anus and/or navel), the weight of the individual, the position of the legs of the individual (e.g., are the legs open or closed), etc. It is noted that some of these factors may change during use and, as such, which portions of the fluid impermeable barrier are contact or spaced portions may change during use.

During use, bodily fluids (e.g., sweat) of the individual may be present between the contact portions of the fluid impermeable barrier and the skin of the individual. For brevity and clarity, the bodily fluids that are present between the contact portions of the fluid impermeable barrier and the skin of the individual are referred to as “trapped fluids.” The trapped fluids may increase the discomfort of using the fluid collection assembly and/or create medical conditions if the trapped fluids are not removed from between the contact portions and the skin of the individual. For example, the trapped fluids may cause rashes, itching, dry skin, dermatitis, fungus growth, etc. Without the fluid flow features, it is difficult to remove the trapped fluids from between the contact portions of the fluid impermeable barrier and the skin of the individual. For example, any possible passageway between the contact portions and the skin of the individual may be blocked or relatively small which may inhibit the flow and, by extension, the removal of the trapped fluids. This problem may be exacerbated when the fluid collection assembly is used with overweight individuals (e.g., individuals having a body mass index greater than 25 or greater than 30). For example, overweight individuals may increase the percentage of the fluid impermeable barrier that are contact portions which, in turn, increases the distance that the trapped fluids must flow to be removed from between the contact portions and the skin of the individual.

However, the fluid flow features formed in the fluid impermeable barrier may allow the trapped fluids to be more easily moved to a location that is not between the contact portions and the skin (e.g., the spaced portions) than if the fluid impermeable barrier does not include the fluid flow features. In other words, the fluid flow features allow the regions between the contact portions and the skin to be more dry than if the fluid impermeable barrier did not include the fluid flow features. Further, removal of the trapped fluids to a location that is not between the contact portions and the skin allows the trapped fluids to be removed from the individual, for example, by evaporation, wiping the individual with an absorbent material, etc.

As will be discussed in more detail below, the fluid flow features may move the trapped fluids to a location that is not between the contact portions and the skin using a variety of methods. For example, the fluid flow features may at least one of form substantially unobstructed paths through which the trapped fluids may flow, provide paths for air flow, or may create forces due to surface tension that move the bodily fluids to a location that is not between the fluid impermeable barrier and the skin.

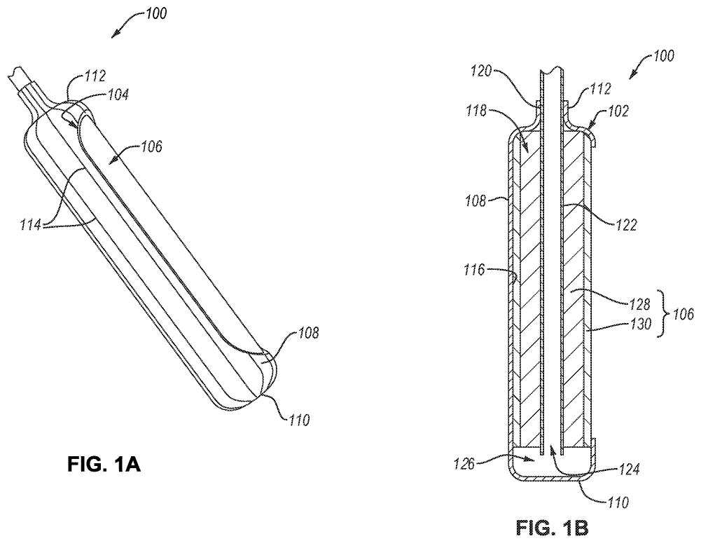

FIG. 1A is a schematic isometric view of a fluid collection assembly 100, according to an embodiment. The fluid collection assembly 100 includes a fluid impermeable barrier 102 defining an opening 104 and at least one porous material 106 extending across the opening 104. The fluid impermeable barrier 102 includes at least one exterior surface 108 extending between a first end 110 and a second end 112 of the fluid impermeable barrier 102. The exterior surface 108 of the fluid impermeable barrier 102 include one or more fluid flow features formed thereon or therein. In the illustrated embodiment, the one or more fluid flow features includes one or more recesses 114 formed in the fluid impermeable barrier 102.

The recesses 114 extend inwardly from the exterior surface 108 of the fluid impermeable barrier 102 and are defined by the fluid impermeable barrier 102. Each of the recesses 114 form passageways that facilitate the removal of trapped fluids that are present between the fluid impermeable barrier 102 and the skin of the individual. For example, during use, it may be difficult to naturally move trapped fluids that are between the exterior surface 108 of the fluid impermeable barrier 102 and the skin of the individual to a location that is not between the fluid impermeable barrier 102 and the skin. The recesses 114 first facilitate the removal of the trapped fluids by decreasing the distance that the trapped fluids must travel before the trapped fluids reach the recesses 114 (i.e., a location that is not directly between the exterior surface 108 of the fluid impermeable barrier 102 and the skin). Second, since each of the recesses 114 form passageways, the trapped fluids may be more easily removed when the trapped fluids are in the recesses 114 than when the trapped fluids are between the exterior surface 108 of the fluid impermeable barrier 102 and the skin.

The recesses 114 may remove the trapped fluids from a location that is between the fluid impermeable barrier 102 and the skin of the individual using any suitable method. In an example, the recesses 114 form airflow passageways that allow air to flow from the spaced portions of the fluid impermeable barrier 102 to the contact portions of the fluid impermeable barrier 102. The airflow may facilitate the removal of vapor of the trapped fluids which, in turn, allows more of the trapped fluids to evaporate. In an example, the recesses 114 may be large enough to allow the trapped fluids to freely flow therein. In such an example, the trapped fluids may flow from a gravimetric high point towards a gravimetric low point of the fluid collection assembly 100. Typically, during use, the second end 112 is the gravimetric high point and the first end 110 is the gravimetric low point. However, the gravimetric high point may be a different than the second end 112 and the gravimetric low point may be different than the first end 110 depending on how the fluid collection assembly 100 is positioned on the individual and/or the orientation of the individual (e.g., is the individual on their back, side, or stomach). In an example, the recesses 114 may form both air flow passageways and allow the trapped fluids to flow therein. In an example, the recesses 114 may remove the trapped fluids from a location that is between the fluid impermeable barrier 102 and the skin of the individual using other methods, such as surface tension, capillary reaction, any other suitable method, or combinations of any of the methods disclosed herein.

Each of the recesses 114 may exhibit a width and a depth. The depth of each of the recesses 114 is measured parallel to a thickness of the fluid impermeable barrier 102 that is measured from the exterior surface 108 to an interior surface 116 (shown in FIG. 1B). The width of each of the recesses 114 is measured perpendicularly to a path (e.g., the longitudinal length of straight path or a slope of a curved path) that the corresponding recess 114 extends and perpendicularly to the depth of the corresponding recess 114. The width and depth of each of the recesses 114 may be about 200 μm to about 400 μm, about 300 μm to about 500 μm, about 400 μm to about 600 μm, about 500 μm to about 800 μm, about 700 μm to about 1 mm, about 900 μm to about 1.2 mm, about 1.1 mm to about 1.4 mm, about 1.3 mm to about 1.6 mm, about 1.5 mm to about 1.8 mm, about 1.7 mm to about 2 mm, about 1.9 mm to about 2.3 mm, about 2.2 mm to about 2.7 mm, about 2.5 mm to about 3 mm, about 2.75 mm to about 3.25 mm, about 3 mm to about 3.5 mm, about 3.25 mm to about 3.75 mm, about 3.5 mm to about 4 mm, about 3.75 mm to about 4.25 mm, about 4 mm to about 4.5 mm, about 4.25 mm to about 4.75 mm, or about 4.5 mm to about 5 mm. It is noted that the width and the depth of each of the recesses 114 may be the same or may be different. It is also noted that at least one of the width or the depth may be substantially constant along at least a portion of the length of the recess 114 or at least one of the width or the depth may vary along at least a portion of the length of the recess 114.

The width and the depth may be selected based on a number of factors. In an embodiment, the width and the depth may be selected based on the method(s) used to remove the trapped fluids from the between the contact portions of the fluid impermeable barrier 102 and the skin. For example, generally, decreasing the width and/or the depth of the recesses 114 may inhibit flow of the trapped fluids in the recesses 114. As such, decreasing the width and/or depth of the recesses 114 may cause removal of the trapped fluids via airflow to be the predominate method for removing the trapped fluids. Whereas, increasing the width and/or depth of the recesses 114 may cause removal of the trapped fluids via flowing the trapped fluids through the recesses 114 to be the predominate method for removing the trapped fluids. The width and/or depth that causes each of these methods of removing the trapped fluids may vary, for example, depending on the composition of the trapped fluids (e.g., is the trapped fluids predominately sweat or another bodily fluid) and the hydration levels of the individual. However, the width and/or depth may cause the method to remove the trapped fluids to be predominately via air flow when the width and/or depth is in the order of micrometers or less than about 1 mm or 2 mm whereas the width and/or depth may cause the method to remove the trapped fluids to be predominately via flowing the trapped fluids through the recesses 114 when the width and/or depth is greater than about 750 μm, greater than about 1 mm, or greater than about 1.5 mm. It is noted that the overlap, as previously discussed, is caused by variations in at least the composition of the trapped fluids and the hydration level of the individual.

In an embodiment, the depth of each of the recesses 114 is selected based on the corresponding width of the recesses 114. For example, increasing the width of the recesses 114 increases the amount of skin that can enter the recess 114. As such, increasing the width of the recesses 114 may require a corresponding increase a depth thereof. Generally, the depth of the recesses 114 is at least 25% the corresponding width of the recesses 114, such as at least 50%, at least 75%, at least 100%, at least 125%, at least 150%, or in ranges of about 25% to about 50%, about 40% to about 60%, about 50% to about 70%, about 60% to about 80%, about 70% to about 90%, about 80% to about 100%, about 90% to about 110%, about 100% to about 120%, about 110% to about 130%, about 120% to about 140%, or about 130% to about 150% the corresponding width of the recesses 114.

In an embodiment, the width and/or depth may be selected based on the weight of the individual. For example, the skin of overweight individuals (e.g., an individual having a body mass index that is greater than 25 or greater than 30) is more likely to enter the recesses 114 than a less overweight individual. As such, the width of the recesses 114 that are configured to be used with overweight individuals may be selected to be smaller than the width of recesses 114 that are configured to be used with individuals that are not overweight. Additionally or alternatively, the depth of the recesses 114 that are configured to be used with overweight individuals may be selected to be greater than the depth of recesses 114 that are configure to be used with individuals that are not overweight.

In an embodiment, as previously discussed, the width and/or depths of the recesses 114 may selectively vary. The width and/or depth of the recesses 114 may vary depending on the relative amount of trapped fluids that the recesses 114 are expected to receive. In an example, the recesses 114 may exhibit the smallest width and/or depth at least one of at or near the opening 104 or at or near a location that is furthest spaced from the first and second ends 110, 112. However, the width and/or depth of the recesses 114 may generally increase with increasing distance from the opening 104 and/or with increasing proximity to the first and second ends 110, 112 since the recesses 114 are likely to collect more trapped fluid with increasing distance from the opening 104 and/or with increasing proximity to the first and second ends 110, 112. In an example, the recesses 114 may exhibit a smallest width and/or depth at a location that is closer to the second end 112 than the first end 110 when the recesses 114 are configured to remove the trapped fluids by flowing the fluids through the recesses 114 and the first end 110 is the gravimetric low point of the fluid collection assembly 100. The width and/or depth of the recesses 114 may then increase with increasing proximity to the first end 110 since the recesses 114 are likely to collect more trapped fluids with increasing proximity to the first end 110.

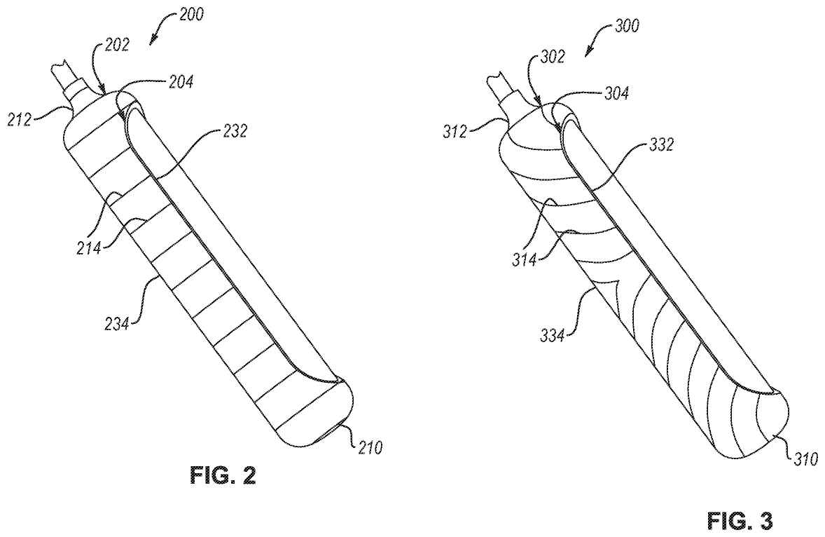

Generally, the recesses 114 extend from contact portions to spaced portions of the fluid impermeable barrier 102. In the illustrated embodiment, the recesses 114 extend longitudinally between the first and second ends 110, 112 of the fluid impermeable barrier 102 since the first and second ends 110, 112 are normally spaced portions of the fluid impermeable barrier 102. Extending the recesses 114 between the first and second ends 110, 112 also allow the fluid collection assembly 100 to be used with overweight individuals and/or individuals who often keep their legs closed since, in such circumstances, the first and second ends 110, 112 may be the only spaced portions of the fluid impermeable barrier 102. It is noted that the recesses 114 do not have to extend longitudinally between the first and second ends 110, 112, as discussed in more detail with regards to FIGS. 2-4.

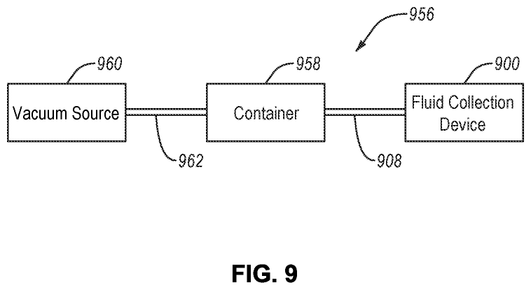

FIG. 1B is a cross-sectional view of the fluid collection assembly 100 shown in FIG. 1A, according to an embodiment. The fluid impermeable barrier 102 may be formed from silicone, another thermosetting polymer, another suitable fluid impermeable material, or combinations thereof. The fluid impermeable barrier 102 defines at least one interior surface 116 opposite the exterior surface 108. The interior surface 116 defines a chamber 118. The opening 104 allows a fluid to enter the chamber 118. For example, when the opening 104 is positioned adjacent to a urethral opening, bodily fluids dispensed from the urethral opening (e.g., urine) enters the chamber 118 through the opening 104. The fluid impermeable barrier 102 also defines an outlet 120, for example, at the second end 112 thereof. The outlet 120 is configured to remove at least some of the bodily fluids that are present in the chamber 118. For example, the fluid collection assembly 100 includes or is in fluid communication with at least one tube 122. The tube 122 is fluidly coupled to the outlet 120, such as positioned through the outlet 120 such that an inlet 124 of the tube 122 is positioned in the chamber 118. As will be discussed in more detail with regards to FIG. 9, the tube 122 may suction the bodily fluids from the chamber 118 and deposit the bodily fluids in a fluid storage container (e.g., fluid storage container 958).

The fluid collection assembly 100 also includes a substantially unoccupied fluid reservoir 126 and, as previously discussed, at least one porous material 106. The porous material 106 may cover at least a portion (e.g., all) of the opening 104. The porous material 106 is exposed to the environment outside of the chamber 118 through the opening 104. The porous material 106 may be configured to wick any fluid away from the opening 104, thereby preventing the fluid from escaping the chamber 118. In the illustrated embodiment, the porous material 106 includes a fluid permeable support 128 and a fluid permeable membrane 130. The fluid reservoir 126, the fluid permeable support 128, and the fluid permeable membrane 130 may be arranged such that any bodily fluid flowing through the opening 104 flows through the fluid permeable membrane 130, through the fluid permeable support 128, and into the fluid reservoir 126. The bodily fluid may then flow from the fluid reservoir 126 to the tube 122 thereby removing the bodily fluid from the chamber 118. In an embodiment, as illustrated, the inlet 124 of the tube 122 may be positioned in or near the fluid reservoir 126 which may facilitate removal of the bodily fluids from the fluid reservoir 126.

The fluid permeable support 128 can be positioned relative to the permeable membrane 130 such that the fluid permeable support 128 maintains the permeable membrane 130 in a particular shape. In an embodiment, the fluid permeable support 128 can be configured to maintain the permeable membrane 130 against or near a urethral opening of a user. For example, the fluid permeable support 128 can include a portion having a curved shape in contact with the permeable membrane 130 such that the permeable membrane 130 is also curved, thus creating a comfortable and secure interface for engagement with the urethral opening and/or the area of the body near the urethral opening (e.g., labia folds).

In an embodiment, the fluid permeable support 128 can be made of a rigid plastic. In an embodiment, the fluid permeable support 128 can have any suitable shape and be formed of any suitable material. For example, the fluid permeable support 128 can be flexible. Additionally, the fluid permeable support 128 can be formed of aluminum, a composite of plastic and aluminum, some other metal, a porous polymer (e.g., nylon, polyester, polyurethane, polyethylene, polypropylene, etc.), an open cell foam, and/or a composite of plastic and another metal. In an embodiment, the fluid permeable support 128 can be formed of a natural material, such as, for example, plant fibers (e.g., Greener Clean manufactured by 3M®), cotton, wool, silk, or combinations thereof. The natural material can include openings that allow fluid to flow through the natural material. In an embodiment, the fluid permeable support 128 can be cylindrical and can define a lumen. In an embodiment, the fluid permeable support 128 can be formed of perforated coated paper, such as tubular waxed paper. In an embodiment, the fluid permeable support 128 may be formed from spun plastic, such as non-woven permeable nylon and/or polyester webbing.

The fluid permeable membrane 130 can be formed of a material that is urine permeable and has wicking properties. The fluid permeable membrane 130 can have a high absorptive rate and a high permeation rate such that urine can be rapidly absorbed by the fluid permeable membrane 130 and/or transported through the fluid permeable membrane 130. In an embodiment, the fluid permeable membrane 130 can be a ribbed knit fabric. In an embodiment, the fluid permeable membrane 130 can include and/or have the moisture-wicking characteristic of gauze (e.g., a silk, linen, or cotton gauze), felt, terrycloth, thick tissue paper, a paper towel, another soft fabric, another smooth fabric, or combinations thereof. In an embodiment, the fluid permeable membrane 130 can be soft and/or smooth (e.g., minimally abrasive) such that the fluid permeable membrane 130 does not irritate the skin of the user (e.g., reduce chaffing). The fluid permeable membrane 130 can be configured to wick fluid away from the urethral opening and/or the skin of the individual such that the dampness of the skin of the user is lessened and infections are prevented. Additionally, the wicking properties of the fluid permeable membrane 130 can help prevent urine from leaking or flowing beyond the assembly onto, for example, a bed. In an embodiment, the fluid permeable membrane 130 can be formed of fine denier polyester fibers coated with a thermoplastic water-based binder system.

In an embodiment, the at least one of the fluid permeable support 128 or the fluid permeable membrane 130 may be omitted from the fluid collection assembly 100 such that the porous material 106 only includes a single material. In an embodiment, the porous material 106 may include three or more materials, such as the fluid permeable support 128, the fluid permeable membrane 130, and at least one additional material. Regardless, one or more components of the porous material 106 may include permeable material designed to wick or pass fluid therethrough. T

In an embodiment, the permeable properties referred to herein may be wicking, capillary action, diffusion, or other similar properties or processes, and are referred to herein as “permeable” and/or “wicking.” Such “wicking” may not include absorption of fluid into the one or more components of the porous material 106. Put another way, substantially no absorption of fluid into the one or more components of the porous material 106 may take place after the porous material 106 is exposed to the bodily fluids. While no absorption is desired, the term “substantially no absorption” may allow for nominal amounts of absorption of fluid into the porous material 106 (e.g., absorbency), such as less than about 10 wt % of the dry weight of the porous material 106, less than about 7 wt %, less than about 5 wt %, less than about 3 wt %, less than about 2 wt %, less than about 1 wt %, or less than about 0.5 wt % of the dry weight of the porous material 106. In an embodiment, the porous material 106 may include an absorbent or adsorbent material

Additional examples of fluid collection assemblies are disclosed in U.S. Pat. No. 10,226,376 filed on Jun. 1, 2017, the disclosure of which is incorporated herein, in its entirety, by this reference.

As previously discussed, when the fluid flow features includes recesses, the recesses do not need to follow the same path as the recesses 114 shown in FIG. 1A. Instead, the recesses may follow any suitable path. FIGS. 2-4 are isometric views of fluid collection assemblies that include one or more recesses following different paths, according to different embodiments. Except as otherwise disclosed herein, the fluid collection assemblies are the same or substantially similar to any of the fluid collection assemblies disclosed herein. For example, the fluid collection assemblies shown in FIGS. 2-4 may include a fluid impermeable barrier defining the one or more recesses and at least one porous material.

Referring to FIG. 2, the fluid collection assembly 200 includes a fluid impermeable barrier 202 extending between a first end 210 and a second end 212 that defines at least one opening 204. The fluid impermeable barrier 202 defines one or more recesses 214. Instead of extending longitudinally from the first end 210 to the second end 212 similar to the recesses 114 of FIG. 1A, the recesses 214 extend circumferentially about the fluid impermeable barrier 202 In other words, the recesses 214 extend in a plane that is generally perpendicular to a longitudinal axis of the fluid impermeable barrier 202. For example, at least some of the recesses extend from an edge 232 of fluid impermeable barrier 202 that defines the opening 204 to an opposing edge (not shown, obscured) of the fluid impermeable barrier 202 that defines the opening 204.

In an embodiment, the fluid collection assembly 200 may be used with individuals who are unlikely to completely or substantially completely enclose the periphery of the fluid impermeable barrier 202. Examples of such individuals includes non-overweight individuals or individuals whom are likely to keep their legs open. When used with such individuals, a back side 234 of the fluid impermeable barrier 202 that is opposite the opening 204 is likely to form a spaced portion of the fluid impermeable barrier 202. Removing the trapped fluids to the back side 234 of the fluid impermeable barrier 202 instead of first or second ends 210, 212 (as is required by the recesses 114 shown in FIG. 1A) may decrease the distance that at least some of the trapped fluids travel to reach a spaced portion of the fluid impermeable barrier 202. The decreased distance may allow for quicker and easier removal of the trapped fluids.

In an embodiment, at least some of the recesses 214 may become less effective when a periphery of the fluid impermeable barrier 202 is completely enclosed, such as when the fluid collection assembly 200 is used with an overweight individual and/or individuals who keeps their legs closed. Completely enclosing the periphery of the fluid impermeable barrier 202 may cause at least some of the recesses 214 to not be in fluid communication with a spaced portion of the fluid impermeable barrier 202. As such, the only means for removing the trapped fluids from the recesses 214 is to deposit the trapped fluid in the opening 204. Restricting the removal of the trapped fluids to the opening 204 may cause the removal of the fluids to be slower than if the recesses 214 where exposed to a spaced portion of the fluid impermeable barrier 202.

Referring to FIG. 3, the fluid collection assembly 300 includes a fluid impermeable barrier 302 extending between a first end 310 and a second end 312 that defines at least one opening 304. The fluid impermeable barrier 302 defines one or more recesses 314. The recesses 314 follow a curved path. For example, the recesses 314 may extend and curve from an edge 332 of the fluid impermeable barrier 302 that defines the opening 304.

In the illustrated embodiment, the recesses 314 may concavely curve relatively to the opening 304. When the recesses 314 are concavely curve, the recesses 314 may initially extend predominately from the edge 332 towards a back surface 334 of the fluid impermeable barrier 302 that is opposite the opening 304. As such, when the back surface 334 is a spaced portion of the fluid impermeable barrier 302, the recesses 314 may initially extend from the edge 332 towards the spaced portions thereby decreasing the distance that any trapped fluids need to travel to reach a spaced portion of the fluid impermeable barrier 302. With increasing distance from the edge 332, the recesses 314 curve towards one of the first or second end 310, 312 (e.g., the closer of the first or second end 310, 312) until the recesses 314 extend predominately towards the first or second end 310, 312. Thus, the recesses 314 are likely to extended to the spaced portions at or near the first and second ends 310, 312 even when the back surface 334 is a contact portion. In other words, the recesses 314 may exhibit at least some of the benefits of the recesses 114, 214 illustrated in FIGS. 1A and 2.

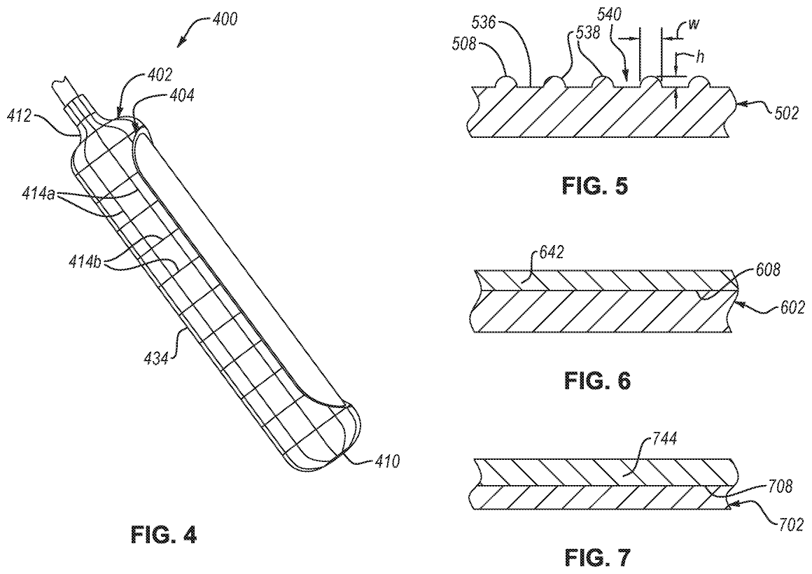

The fluid impermeable barriers disclosed herein may define recesses having paths that are different than the paths illustrated in FIGS. 1A, 2, and 3. For example, the recesses may follow a spiral path, a zig-zag path, a convexly curved path relative to the opening, or any other suitable path. Further, the fluid impermeable barriers disclosed herein may also define a combination of any of the recesses disclosed herein. For example, referring to FIG. 4, the fluid collection assembly 400 a fluid impermeable barrier 402 extending between a first end 410 and a second end 412 that defines an opening 404. The fluid impermeable barrier 402 defines a plurality of recesses therein, such as one or more first recesses 414a and one or more second recesses 414b. The first recesses 414a may be the same or substantially similar to the recesses 114 shown in FIG. 1A. For example, the first recesses 414a may extend longitudinally between the first and second ends 410, 412 of the fluid impermeable barrier 402. The second recesses 414b may be the same or substantially similar to the recesses 214 shown in FIG. 2. For example, the second recesses 414b may extend circumferentially about the fluid impermeable barrier 402 In other words, the second recesses 414b may extend perpendicularly or substantially perpendicularly to the first recesses 414a.

The first and second recesses 414a, 414b intersect with each other to form interconnected recesses. The interconnected recesses allow the fluid collection assembly 400 to exhibit the benefits of both the recesses 114, 214 illustrated in FIGS. 1A and 2. For example, the second recesses 414b may decrease the distance that some trapped fluids need to travel when a back surface 434 of the fluid impermeable barrier 402 opposite the opening 404 is a spaced portion of the fluid impermeable barrier 402. However, if the back surface 434 of the fluid impermeable barrier 402 is a contact portion, the first recesses 414a fluidly couple the trapped fluids to the first and second ends 410, 412.

The fluid flow features disclosed herein may include other features instead of or in conjunction with the recesses disclosed herein. For example, FIGS. 5-7 are schematic cross-sectional views of a portion of a fluid collection assembly that includes fluid flow features other than recesses, according to different embodiments. Except as otherwise disclosed herein, the fluid collection assemblies illustrated in FIGS. 5-7 are the same or substantially similar to any of the fluid collection assemblies disclosed herein. For example, the fluid collection assemblies may include at least one porous material and a fluid impermeable barrier defining an opening, outlet, and chamber. Further, as previously discussed, any of the fluid flow features illustrate in FIGS. 5-7 may be used instead of or in conjunction with any of the other fluid flow features disclosed herein.

Referring to FIG. 5, the fluid collection assembly 500 includes a fluid impermeable barrier 502 and one or more fluid flow features. The fluid flow features includes a plurality of protrusions 538 formed in the fluid impermeable barrier 502. For example, the fluid impermeable barrier 502 may define a base surface 536 and the protrusions 538 extend outwardly from the base surface 536. The portion of the protrusions 538 that is furthest from the base surface 536 may form the exterior surface 508 of the fluid impermeable barrier 502. The base surface 536 and the protrusions 538 may form a plurality of interconnected channels 540 therebetween. Similar to the recesses discussed above, the channels 540 may allow the trapped fluids to move from a contact portion of the fluid impermeable barrier 502 to a spaced portion of the fluid impermeable barrier 502, for example, using airflow to remove vapor of the trapped fluids or allowing the trapped fluids to flow therein.

Each of the protrusions 538 exhibits a width “w” and a height “h.” The width “w” and height “h” of each of the protrusions 538 may be about 200 μm to about 400 μm, about 300 μm to about 500 μm, about 400 μm to about 600 μm, about 500 μm to about 800 μm, about 700 μm to about 1 mm, about 900 μm to about 1.2 mm, about 1.1 mm to about 1.4 mm, about 1.3 mm to about 1.6 mm, about 1.5 mm to about 1.8 mm, about 1.7 mm to about 2 mm, about 1.9 mm to about 2.3 mm, about 2.2 mm to about 2.7 mm, about 2.5 mm to about 3 mm, about 2.75 mm to about 3.25 mm, about 3 mm to about 3.5 mm, about 3.25 mm to about 3.75 mm, about 3.5 mm to about 4 mm, about 3.75 mm to about 4.25 mm, about 4 mm to about 4.5 mm, about 4.25 mm to about 4.75 mm, or about 4.5 mm to about 5 mm. It is noted that the width “w” and the height “h” of each of the protrusions 538 may be the same or may be different.

The width “w,” height “h,” and the number of protrusions 538 (i.e., the number of protrusions in a selected surface area) may be selected based on a number of factors. In an embodiment, the width “w,” height “h,” and/or number of protrusions 538 may be selected based on the method(s) used to remove the trapped fluids from the between the contact portions of the fluid impermeable barrier 102 and the skin. For example, generally, decreasing the width “w,” decreasing the height “h,” and/or increasing the number of protrusions 538 may cause removal of the trapped fluids via airflow to be the predominate method for removing the trapped fluids. Whereas, increasing the width “w,” increasing the height “h,” and/or decreasing the number of protrusions 538 may cause removal of the trapped fluids via flowing the trapped fluids through the protrusions 538 to be the predominate method for removing the trapped fluids. The width “w,” height “h,” and the number of protrusions 538 that causes each of these methods of removing the trapped fluids may vary, for example, depending on the composition of the trapped fluids and the hydration levels of the individual. However, the width “w” and/or height “h” may cause the method to remove the trapped fluids to be predominately via air flow when the width “w” and/or height “h” is in the order of micrometers or less than about 1 mm or 2 mm whereas the width “w” and/or height “h” may cause the method to remove the trapped fluids to be predominately via flowing the trapped fluids through the protrusions 538 when the width “w” and/or height “h” is greater than about 750 μm, greater than about 1 mm, or greater than about 1.5 mm.

In an embodiment, width “w,” height “h,” or the number of protrusions 538 is selected based on another of width “w,” height “h,” or the number of protrusions 538. For example, decreasing the number of protrusions 538 may increase the amount of skin that can enter the channels 540. As such, decreasing the number of protrusions 538 may require a corresponding increase the width “w” and/or the height “h” of the protrusions 538.

In an embodiment, width “w,” height “h,” and/or the number of protrusions 538 may be selected based on the weight of the individual. For example, the skin of overweight individuals (e.g., an individual having a body mass index that is greater than 25 or greater than 30) is more likely to enter the channels 540 than a less overweight individual. As such, the width “w” of the protrusions 538 that are configured to be used with overweight individuals may be selected to be greater than the width “w” of protrusions 538 that are configured to be used with individuals that are not overweight. Additionally or alternatively, the height “h” of the protrusions 538 that are configured to be used with overweight individuals may be selected to be greater than the height “h” of protrusions 538 that are configure to be used with individuals that are not overweight. Additionally or alternatively, the number of protrusions 538 may be greater when the fluid collection assembly that includes the fluid impermeable barrier 502 is configured to be used with overweight individuals instead of individuals who are not overweight.

Referring to FIG. 6, the fluid collection assembly 600 includes a fluid impermeable barrier 602 defining at least one exterior surface 608. The fluid collection assembly 600 also includes one or more fluid flow features. The fluid flow features includes a hydrophobic layer 642 disposed on and attached to at least a portion of the exterior surface 608. The hydrophobic layer 642 causes any of the trapped fluid that come in contact therewith to form droplets instead of wetting the surface. Forming the droplets causes the hydrophobic layer 642 to be spaced from the skin of the individual thereby allowing airflow between the hydrophobic layer 642 and the skin. As previously discussed, the air flow may remove vapor of the trapped fluids. Further, any force that compresses the droplets of the trapped fluids is likely to also apply a force to the droplets that accelerates movements of the droplet from between a contact portion of the hydrophobic layer 642 to a spaced portion of the hydrophobic layer 642.

In an embodiment, the hydrophobic layer 642 exhibits a contact angle with water that is at least greater than the contact angle between the exterior surface 608 of the fluid impermeable barrier 602 and water. In an embodiment, the hydrophobic layer 642 exhibits a contact angle with water that is greater than about 90°, greater than about 105°, greater than about 120°, greater than about 135°, greater than about 150°, greater than about 165°, about 180°, or in ranges of about 90° to about 120°, about 105° to about 135°, about 120° to about 150°, about 135° to about 165°, or about 150° to about 180°.

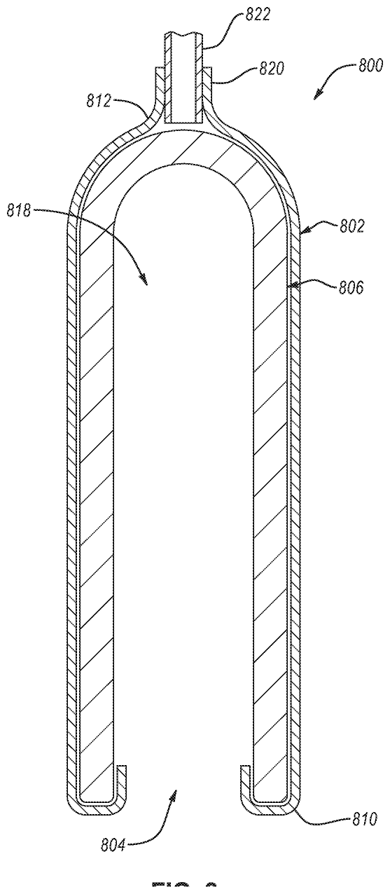

Referring to FIG. 7, the fluid collection assembly 700 includes a fluid impermeable barrier 702 defining at least one exterior surface 708. The fluid collection assembly 700 also includes one or more fluid flow features. The fluid flow features include a wicking layer 744 disposed on and attached to at least a portion of the exterior surface 708. The wicking layer 744 may form airflow passages that facilitate removal of the trapped fluids. The wicking layer 744 may also receive and redistribute any trapped fluid received thereby removing the trapped fluids from the skin. For example, the wicking layer 744 may remove trapped fluids from a contact portion thereof and redistribute the trapped fluids to a spaced portion thereof. The wicking layer 744 may be formed from any suitable material, such as any of the porous materials disclosed herein.

The features and principles regarding the fluid collection assemblies disclosed above may also be applied to a fluid collection assembly that is configured to receive urine from a male urethral opening. For example, FIG. 8 is a schematic cross-sectional view of a fluid collection assembly 800 that is configured to received urine from a male urethral opening, according to an embodiment. Except as otherwise disclosed herein, the fluid collection assembly 800 is the same or substantially similar to any of the fluid collection assemblies disclosed herein. For example, the fluid collection assembly 800 includes a fluid impermeable barrier 802 that defines a chamber 818 and at least one porous material 806. The fluid collection assembly 800 may also include at least one tube 822 that is in fluid communication with the chamber 818.

The fluid impermeable barrier 802 includes a first end 810 and an opposing second end 812. The first end 810 of the fluid impermeable barrier 802 defines an opening 804 and the second end 812 of the fluid impermeable barrier 802 may define an outlet 820 that is in fluid communication with the tube 822 (e.g., the tube 822 is positioned through the outlet 820). The opening 804 and the outlet 820 provide access to the chamber 818. As illustrated, the opening 804 is configured to receive the male urethral opening (e.g., the penis) such that the male urethral opening is disposed in the chamber 818. However, it is noted that, in some embodiments, the opening 804 may be configured to be positioned adjacent to the male urethral opening instead of receiving the male urethral opening.

Additional examples of fluid collection assemblies that are configured to receive bodily fluids from a male urethral opening that may include the features and principles disclosed herein are disclosed in U.S. patent application Ser. No. 16/433,773 filed on Jun. 6, 2019, the disclosure of which is incorporated herein, in its entirety, by this reference.

FIG. 9 is a schematic illustration of fluid collection system 956 that includes a fluid collection assembly 900, according to an embodiment. The fluid collection assembly 900 may include any of the fluid collection assemblies disclosed herein. The fluid collection assembly 900 may be in fluid communication with a fluid storage container 958 via at least one first tube 922 (e.g., tube 122, 822 of FIGS. 1B and 8). The fluid storage container 958 is positioned downstream from the fluid collection assembly 900. The fluid storage container 958 may be in fluid communication with a vacuum source 960 via at least one second tube 962. The vacuum source 960 is positioned downstream from the fluid storage container 958. During operation, the vacuum source 960 provides a suction force to the fluid collection assembly 900. The suction force draws fluid into the chamber and towards the first tube 922. The fluid that enters the first tube 922 is pulled by the suction force towards the fluid storage container 958 such that the fluid storage container 958 receives the fluid. The fluid storage container 958 may be configured to inhibit the fluid from flowing from the fluid storage container 958 to the vacuum source 960.

While various aspects and embodiments have been disclosed herein, other aspects and embodiments are contemplated. The various aspects and embodiments disclosed herein are for purposes of illustration and are not intended to be limiting.

Terms of degree (e.g., “about,” “substantially,” “generally,” etc.) indicate structurally or functionally insignificant variations. In an example, when the term of degree is included with a term indicating quantity, the term of degree is interpreted to mean±10%, ±5%, +2% or 0% of the term indicating quantity. In an example, when the term of degree is used to modify a shape, the term of degree indicates that the shape being modified by the term of degree has the appearance of the disclosed shape. For instance, the term of degree may be used to indicate that the shape may have rounded corners instead of sharp corners, curved edges instead of straight edges, one or more protrusions extending therefrom, is oblong, is the same as the disclosed shape, etc.

1. A fluid collection assembly, comprising;
a fluid impermeable barrier defining a chamber, at least one opening configured to be positioned a least proximate to a urethral opening of an individual, and an outlet; the fluid impermeable barrier including at least one exterior surface and at least one interior surface defining the chamber; and
one or more fluid flow features at least one of defined by, formed by, or disposed on at least a portion of the fluid impermeable barrier or on at least a portion of the at least one exterior surface of the fluid impermeable barrier, the one or more fluid flow features configured to remove trapped fluids between the at least one exterior surface and skin of an individual, the one or more fluid flow features including at least one of:
a plurality of recesses extending inwardly from the at least one exterior surface;
a plurality of protrusions extending outwardly from a base surface of the fluid impermeable barrier;
a hydrophobic layer disposed on at least a portion of the at least one exterior surface; or
a wicking layer disposed on at least a portion of the at least one exterior surface.

2. The fluid collection assembly of claim 1, wherein the at least one opening is an elongated opening.

3. The fluid collection assembly of claim 1, wherein the one or more fluid flow features include the plurality of recesses.

4. The fluid collection assembly of claim 3, wherein:
the fluid impermeable barrier includes a first end and a second end opposite the first end; and
at least one of the plurality of recesses extends between the first end of the fluid impermeable barrier and the second end of the fluid impermeable barrier.

5. The fluid collection assembly of claim 3, wherein at least one of the plurality of recesses extends circumferentially about the fluid impermeable barrier.

6. The fluid collection assembly of claim 3, wherein at least one of the plurality of recesses is curved.

7. The fluid collection assembly of claim 6, wherein the at least one of the plurality of recesses is concavely curved relative to the at least one opening.

8. The fluid collection assembly of claim 3, wherein at least some of the plurality of recesses intersect with each other.

9. The fluid collection assembly of claim 1, wherein the one or more fluid flow features includes the plurality of protrusions.

10. The fluid collection assembly of claim 1, wherein the one or more fluid flow features includes the hydrophobic layer.

11. The fluid collection assembly of claim 1, wherein the one or more fluid flow features includes the wicking layer.

12. The fluid collection assembly of claim 1, further comprising at least one porous material disposed in the chamber.

13. The fluid collection assembly of claim 1, further comprising an substantially unoccupied reservoir located at an end of the fluid impermeable barrier.

14. A method of using a fluid collection assembly, the method comprising:
positioning at least one opening defined by a fluid impermeable barrier of the fluid collection assembly to be at least proximate to a urethral opening of an individual, the fluid impermeable barrier defining an outlet, the fluid impermeable barrier including at least one exterior surface and at least one interior surface defining a chamber, wherein the at least one opening and the outlet provide access to the chamber; and
with one or more fluid flow features, removing trapped fluids from between the at least one exterior surface of the fluid impermeable barrier and skin of the individual to a location that is different from between the at least one exterior surface of the fluid impermeable barrier and the skin of the individual, wherein the one or more fluid flow features are at least one of defined by, formed by, or disposed on at least a portion of the fluid impermeable barrier or on at least a portion of the at least one exterior surface, the one or more fluid flow features including at least one of:
a plurality of recesses extending inwardly from the at least one exterior surface;
a plurality of protrusions extending outwardly from a base surface of the fluid impermeable barrier;
a hydrophobic layer disposed on at least a portion of the at least one exterior surface; or
a wicking layer disposed on at least a portion of the at least one exterior surface.

15. The method of claim 14, wherein:
the one or more fluid flow features includes the one or more recesses; and
removing trapped fluids from between the at least one exterior surface of the fluid impermeable barrier and skin of the individual to a location that is different from between the at least one exterior surface of the fluid impermeable barrier and the skin of the individual includes flowing air through the one or more recesses to remove vapor of the trapped fluids.

16. The method of claim 14, wherein:
the one or more fluid flow features include the one or more recesses; and
removing trapped fluids from between the at least one exterior surface of the fluid impermeable barrier and skin of the individual to a location that is different from between the at least one exterior surface of the fluid impermeable barrier and the skin of the individual includes at least one of flowing air through the one or more recesses or flowing the trapped fluids through the one or more recesses.

17. The method of claim 14, wherein:
the one or more fluid flow features include the plurality of protrusions; and
removing trapped fluids from between the at least one exterior surface of the fluid impermeable barrier and skin of the individual to a location that is different from between the at least one exterior surface of the fluid impermeable barrier and the skin of the individual includes at least one of flowing air through one or more interconnected channels formed between the plurality of protrusions or flowing the trapped fluids through the one or more interconnected channels.

18. The method of claim 14, wherein the one or more fluid flow features includes the hydrophobic layer.

19. The method of claim 14, wherein the one or more fluid flow features includes the wicking layer; and
removing trapped fluids from between the at least one exterior surface of the fluid impermeable barrier and skin of the individual to a location that is different from between the at least one exterior surface of the fluid impermeable barrier and the skin of the individual includes at least one of flowing air through one or more interconnected channels formed in the wicking layer or flowing the trapped fluids through the one or more interconnected channels.

20. A fluid collection system, comprising:
a fluid collection assembly including:
a fluid impermeable barrier defining a chamber, at least one opening configured to be positioned a least proximate to a urethral opening of an individual, and an outlet, the fluid impermeable barrier including at least one exterior surface and at least one interior surface defining the chamber; and
one or more fluid flow features at least one of defined by, formed by, or disposed on at least a portion of the fluid impermeable barrier or on at least a portion of the at least one exterior surface of the fluid impermeable barrier, the one or more fluid flow features configured to at least one of remove trapped fluids between the at least one exterior surface and skin of the individual, the one or more fluid flow features including at least one of:
a plurality of recesses extending inwardly from the at least one exterior surface;
a plurality of protrusions extending outwardly from a base surface of the fluid impermeable barrier;
a hydrophobic layer disposed on at least a portion of the at least one exterior surface; or
a wicking layer disposed on at least a portion of the at least one exterior surface;
a fluid storage container;
a vacuum source configured to apply a suction force; and
at least one tube that places the chamber, the fluid storage container, and the vacuum source in fluid communication with each other.
